(19) United States
(12) Patent Application Publication
(10) Pub. No.: US 2008/0187506 A1
(43) Pub. Date: Aug. 7, 2008

(54) Hair care composition

(76) Inventors: Jose Antonio Carballada, Cincinnati, OH (US); Timothy Roy Nijakowski, Mason, OH (US); Bryan Patrick Murphy, Loveland, OH (US); Alexis M.J.A. Huyghues-Despointes, Caracus (VE); Axel Kalbfleisch, Darmatadt (DE)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/702,308

(22) Filed: Feb. 5, 2007

(51) Int. Cl. A61Q 5/12; A61K 8/72

(52) U.S. Cl. 424/70.12; 424/70.11

(57) ABSTRACT

Hair care composition comprising from about 0.1% to about 20% of a sulfopropyl(meth)acrylate compound, an alpha methylene lactone, or a mixture thereof, wherein the sulfopropyl(meth)acrylate compound and the alpha methylene lactone each have a molecular weight of less than 500 grams/mole; and a dermatologically acceptable carrier.

### FIELD OF THE INVENTION

The present invention relates to hair care compositions comprising a sulfopropyl(meth)acrylate compound and/or an alpha methylene lactone. The present invention further relates to a method of chemically modifying the internal region of a hair shaft. The present invention further relates to a kit comprising a first composition comprising a sulfopropyl(meth)acrylate compound and/or an alpha methylene lactone, and a second composition comprising an initiator.

### BACKGROUND OF THE INVENTION

Hair style formation and retention traditionally has been accomplished by the use of styling products comprising polymers and other components that at least partially coat the hair and act on the surface of hair fibers. Several disadvantages are associated with this approach. Loss of hair style due to passage of time, elevated humidity, excessive motion, etc. may cause a consumer to feel the need to refresh a hair style throughout the day. This often requires application of additional styling product(s). The benefits of traditional styling products also may be diminished when applied to hair to which conditioning agents also have been applied. In addition, application of materials to the surface of hair may compromise the natural feel and appearance of the hair and result in a dull appearance and/or a stiff or otherwise unpleasant feel.

A need exists, therefore, to provide hair care compositions that avoid the aforementioned disadvantages, and that provide improved shape retention and style durability, with or without the use of a hair conditioning agent. Such compositions may be especially desirable for consumers seeking a hair style with increased volume.

### SUMMARY OF THE INVENTION

The present invention meets the aforementioned need by providing hair care compositions comprising monomers that penetrate the hair and that bind to an internal portion of the hair shaft. Thus the internal structure of the hair is modified, and results in increased rigidity of the hair shaft. Without wishing to be limited by theory, hair is comprised of an outer layer, or cuticle, which in turn comprises overlapping cuticle cells that serve to protect the inner hair shaft. The inner hair shaft, or cortex, comprises cortical cells. Both cuticle and cortical cells are made of proteins which are crosslinked and arranged in specific patterns. The proteins of the cuticle and cortical cells may play a role in controlling the mechanical properties of hair, for example, rigidity, strength, and elasticity. Applicants believe that selected monomers having a relatively low molecular weight are able to penetrate the cuticle and cortex and bind to both reduced keratinous proteins and to each other. Thus, rather than applying a pre-formed polymer to the outer layer of the hair, the present invention describes applying relatively small molecules that are able to penetrate into the internal region of the hair shaft, where the molecules may bond to the hair and/or to each other to form larger molecules. This, in turn, internally increases the rigidity of the hair, which allows style formation of increased volume and style retention for longer periods of time.

The present invention offers several advantages over other approaches. Because the complexes formed by the monomers with the keratinous proteins remain stable for an extended period of time, hair treated with the compositions of the present invention may continue to exhibit increased rigidity after multiple washings and/or wetting of the hair. The hair further exhibits fewer negative effects of moisture and/or humidity, such as frizziness or limpness. The hair also may be easier to style, may require application of less styling product, and/or may hold the style for longer periods of time.

The following describe some non-limiting embodiments of the present invention.

According to a first embodiment of the present invention, a hair care composition is provided, comprising from about 0.1% to about 20% of a sulfopropyl(meth)acrylate compound, an alpha methylene lactone, or a mixture thereof, wherein the sulfopropyl(meth)acrylate compound and the alpha methylene lactone each have a molecular weight of less than 500 grams/mole; and a dermatologically acceptable carrier.

According to another embodiment of the present invention, a method of chemically modifying the internal region of a hair shaft is provided, comprising the step of applying to hair a first composition according to the first embodiment and the step of applying to the hair an initiator.

According to yet another embodiment of the present invention, a kit is provided comprising a first composition according to the first embodiment; and a second composition comprising an initiator; wherein the first composition and the second composition are separately packaged within the kit.

According to yet another embodiment of the present invention, an article of commerce is provided comprising a first composition according to the first embodiment; a second composition comprising an initiator, and advertisement material pertaining to the compositions. The advertisement material may comprise images comparing the appearance of a person prior to use of the compositions to the appearance of the same person after use of the composition.

According to yet another embodiment of the present invention, a method of marketing a kit comprising a first composition according to the first embodiment; and a second composition comprising an initiator, wherein the method of marketing comprises the step of making available to a consumer the kit, and providing a communication to the consumer that the compositions may provide one or more benefits to the hair, including but not limited to increased style retention, increased style durability, increased appearance of volume, increased resistance to moisture, and combinations thereof.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

“Hair,” as used herein, means hair on the human head and scalp. “Hair shaft” means an individual hair, and may be used interchangeably with the term “hair.”

“Internal region of the hair shaft,” as used herein, means any non-surface portion of the hair shaft, including the inner portion of the cuticle. “Non-surface portion” may be understood to mean that portion of the hair that is not in direct contact with the outside environment.

“Proximal to the scalp,” as used herein, means that portion of an extended, or substantially straightened, hair shaft that is closer in distance to the scalp than to the end of the hair. Thus, about 50% of the hair would be considered proximal to the scalp, and about 50% of the hair would be distal to the scalp. “x cm proximal to the scalp” means a distance “x” along the hair, with one endpoint being on or directly adjacent to the scalp, and the second endpoint being measured “x” centimeters along the length of the extended or substantially straightened hair.

“Dermatologically-acceptable carrier,” as used herein, that the compositions or components described are suitable for use in contact with human keratinous tissue without undue toxicity, incompatibility, instability, allergic response, and the like.

“Derivatives,” as used herein, includes but is not limited to, amide, ether, ester, amino, carboxyl, acetyl, and/or alcohol derivatives of a given compound.

“Monomer,” as used herein, means a discrete, non-polymerized chemical moiety capable of undergoing polymerization in the presence of an initiator.

“Ethylenic monomer,” as used herein, means a chemical species that contains an olefenic carbon-carbon double bond (C═C) and is capable of undergoing polymerization in the presence of an initiator.

“Multivalent cation,” as used herein, means an element having a net ionic charge of from +2 to +7.

“Chemically modify,” or grammatical equivalents thereof, as used herein, means that a chemical moiety such as monomer and/or crosslinker, stably affixes to a second chemical moiety, for example, a keratin protein, another component of hair, and/or another monomer or crosslinker.

“Stably affix” is understood to include both covalent and non-covalent forms of chemical bonds that once formed, remain unchanged through wetting, washing, styling and other types of hair treatment. In general, stably affixed chemical moieties may not be removed from the hair without damaging or substantially destroying the hair.

“Bending rigidity,” as used herein, is determined by the Kato Single Fiber Bending Method. One example of determining bending rigidity is as follows: Select 6 individual hairs from an area to which the composition(s) of the present invention will be applied. Mount a brass ferrule to one end of an individual fiber using a RS Components Press—Sock No. 622-032. Cut the hairs to a length of 55 mm from the base of the ferrule. Using a KES-FB2-SH Single Fiber Bending Tester System (Kato Tech Co., LTD) run by KES-FB system measurement program version 6.35WE, run “Single Hair Fiber Bending Method” using the following settings: Sample=Hair; Sensitivity=2.5; Count=1; Curvature=2.5; Repetition=1. Load the sample as follows: Lock the back chuck, set check switch on OSC and confirm a meter reading of ÷10V. Settings: Sensitivity (initially)=0.08 gf/10V; speed switch=1; speed dial=2. Place the hair sample end without the brass ferrule in the rear chuck, centered vertically. Very lightly tighten rear chuck with knob on side. Pull fiber through the front chuck, ensuring fiber is straight, not taut, and not loose. Tighten chuck with knob. Unlock chuck holder and shut the enclosure. Perform measurement as follows: Set check knob to BAL and obtain a reading of 0V on the voltmeter. Set check knob to MES and obtain a reading of 0V on the voltmeter. Measure each hair 3 times, reloading the sample prior to each evaluation, to obtain a total of 18 readings for the set of six hair samples. Average the 18 readings to obtain a measurement of the “bending moment” in gf·cm/fiber, where “gf” means grams of force, for the set of samples. (A typical bending moment value is approximately 0.2 gf·cm/fiber.) Repeat the procedure for the same set of samples after application, as described herein, of the composition(s) of the present invention. The bending rigidity is increased if the average bending moment of the same set of samples after treatment described in the present invention is at least 20% greater than the average bending moment of the initial measurement.

“Curl retention” may be measured as follows: Using a swatch of hair weighing approximately 1 g and having a length of about 10 inches, thoroughly wet the hair with water and comb sufficiently to detangle. Affix the top portion of the hair, for example with a rubber band, to a ceramic rod having a length of 6 inches and a diameter of 0.5 inches. Wrap the hair around the rod in a spiral fashion with substantially no overlap of hair per spiral and with no space between each spiral. Stop wrapping around the rod when approximately 2 inches of hair are remaining, which are then enclosed with folded end papers as one of skill in the art would understand to apply to permed hair. Continue curling the wrapped last two inches around the ceramic rod and attach the bottom portion of the hair to the rod using adhesive tape which contacts the rod and endpaper only. Allow the hair to set in this curled position for 8-20 hours or until dry. Remove the hair swatch from the rod and hang vertically. Optionally, the hair may be hung in an environment having a temperature of about 32° C. and about 80% relative humidity. Measure the total length of the curled hair from top to bottom, and record as the length at time zero (length_{(time 0)}). Repeat the measurement after 60 minutes and record as the total length of the hair at time 60 (length_{(time 60)}). Calculate the curl retention number (CRN) as:

Curl Retention Number_{(time 60)}=[Length_{(time 60)}−Length_{(time 0)}]/[Length_{(time 0)}]

Repeat the method with four additional swatches, and average the five measurements of the difference in total length to obtain the average Curl Retention Number. “Increased curl retention” means that the average curl retention number of a set of five swatches of hair to which the composition(s) of the present invention have been applied is at least 20% less than the average curl retention number of a control set of five swatches, treated according to the same method but to which no composition(s) of the present invention have been applied.

“Increased style retention and/or durability,” as used herein, means that the hair style formed, shaped, and/or obtained after application of the composition of the present invention to the hair is maintained for a longer period of time relative to hair of the same being to which no composition has been applied.

“Increased appearance of volume,” as used herein, means that the hair exhibits a visually noticeable greater volume, i.e., distance between the scalp and the outermost layer of hair style and/or distance between individual hairs, after application of a composition of the present invention relative to before a composition was applied.

“Increased resistance to moisture,” as used herein, means that after application of a composition of the present invention, the hair fails to exhibit visually noticeable effects, such as loss of volume, loss of style, increase in frizz, etc. upon exposure to water vapor (i.e., relative humidity greater than 50%) relative to before a composition has been applied.

“Reserve alkalinity,” as used herein, means the relative strength and apparent concentration of the base used to adjust the pH, measured as described in ASTM D 1121, wherein the base of the present invention is substituted for the antifreeze used in the method.

“Kit,” as used herein, means a packaging unit comprising a first composition and a separately packaged second composition, as described herein.

“Separately packaged,” as used herein, means any form of packaging that prevents a first composition from coming into physical contact, or admixing, with a second composition.

“Separately packaged” may mean that the individual compositions are packaged in separate containers, or alternatively in a single container partitioned such that the compositions are not in physical contact.

“Implement,” as used herein, means a device used to facilitate application of a composition to the hair and/or manipulation of the hair. Examples of implements include, but are not limited to, a comb, a means for directed delivery (e.g., an applicator or tube), a covering for the hair (e.g., plastic bag, shower cap, etc.), and combinations thereof.

“Energy delivery device,” as used herein, means any device used to deliver energy to keratinous tissue, including the hair and scalp. “Delivery of energy,” means that the surface of the keratinous tissue is exposed to the energy emanating from the energy delivery device, where it may penetrate to the desired layers of the tissue, including the hair shaft and/or hair follicle. Energy includes but is not limited to energy in the form of light, heat, sound (including ultrasonic waves), electrical energy, magnetic energy, electromagnetic energy (including radiofrequency waves and microwaves), and combinations thereof.

### I. Compositions

The present invention comprises a composition comprising a sulfopropyl(meth)acrylate compound and/or an alpha methylene lactone. When referring to an embodiment that contemplates more than one composition, the composition comprising a sulfopropyl(meth)acrylate compound and/or an alpha methylene lactone may be referred to as a “first composition.” The composition may have a pH of about 7.0 and below, alternatively of from about 2.0 to about 7.0, and alternatively of from about 3.0 to about 6.0. In all embodiments, “about,” when used in reference to pH, is understood to mean ±0.1 pH unit.

“Sulfopropyl(meth)acrylate compound,” as used herein, is understood to include derivatives, salts and/or isomers of sulfopropyl(meth)acrylate. The sulfopropyl(meth)acrylate compounds of the present invention are understood to be in a non-polymerized form, and do not include polymers of sulfopropyl(meth)acrylate. One example of a useful sulfopropyl(meth)acrylate compound is 3-sulfopropyl(meth)acrylate, CAS #31098-20-1. Sulfopropyl(meth)acrylate compounds also may be known as sulfopropyl acrylate esters; sulfoalkylpropenoate; acrylic acid propyl ester sulfonates; propenoic acid sulfoalkyl ester; and/or sulfoalkylpropenoic acid ester. The composition may comprise from about 0.1% to about 20%, alternatively from about 1% to about 15%, alternatively from about 5% to about 10%, alternatively from about 0.1% to about 10%, and alternatively from about 10% to about 20% of a sulfopropyl(meth)acrylate compound.

“Alpha methylene lactone,” as used herein, is understood to include derivatives, salts and/or isomers of a compound having the general structure:

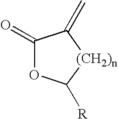

where n is a positive integer having a value from 1 up to and including 6, alternatively from 1 up to and including 4, and alternatively is 1; and where R is hydrogen or a C1-C4 alkyl, hydroxyalkyl, alkylhalide, alkylether, allyl, or aryl moiety. One example of a useful alpha methylene lactone is alpha-methylene-gamma-butyrolactone, CAS #547-65-9. Other examples of useful alpha methylene lactones include, but are not limited to, alpha-methylene-gamma-valerolactone, alpha-methylene-epsilon-caprolactone, 3-methylideneoxam-2-one, (5S)-S-(hydroxymethyl)-3-methylidene-oxolam-2-one, 3-methylidene-1-oxaspiro-[4,5]-decan-2-one, and mixtures thereof. The alpha methylene lactones of the present invention are understood to be in a non-polymerized form, and do not include polymers of alpha methylene lactones. The composition may comprise from about 0.1% to about 20%, alternatively from about 1% to about 15%, alternatively from about 5% to about 10%, alternatively from about 0.1% to about 10%, and alternatively from about 10% to about 20%, of an alpha methylene lactone.

The sulfopropyl(meth)acrylate and the alpha methylene lactone compounds are of a size suitable to penetrate the hair shaft, and have a molecular weight of 500 g/mole or less, alternatively from about 100 g/mole to about 500 g/mole, alternatively from about 100 g/mole to about 400 g/mole, and alternatively from about 200 g/mole to about 400 g/mole, where “g/mole” means grams per mole of a compound.

The composition further may comprise at least one ethylenic monomer having a size suitable to penetrate the hair shaft and a molecular weight of 500 g/mole or less, alternatively from about 50 g/mole to about 500 g/mole, alternatively from about 75 g/mole to about 400 g/mole, and alternatively from about 100 g/mole to about 400 g/mole. Examples of ethylenic monomers suitable for use in the first composition of the present invention include, but are not limited to mesaconic acid, 2-pentenoic acid, tiglic acid, tiglic acid esters, furan-3-acrylic acid, 2-acrylamido-2-methyl-1-propanesulfonic acid, maleamic acid, 3-aminocrotonic acid, crotonic acid esters, itaconic anhydride, trimethylsilylacrylate, poly(ethyleneglycol)acrylates, N-vinylacetamide, 2-acetamidoacrylic acid, vinylsulfonic acid, tetrahydrofurfurylacrylate, N-methyl-N-vinylacetamide, vinylpropionate, vinylanisole, vinylcrotonate, methyl 3-hydroxy-2-methylenebutyrate, and salts, isomers, derivatives and mixtures thereof. The composition may comprise from about 0.1% to about 20%, alternatively from about 1% to about 15%, alternatively from about 5% to about 10%, alternatively from about 0.1% to about 10%, and alternatively from about 10% to about 20%, of an ethylenic monomer.

In one embodiment, the ratio of the weight percentage of the sulfopropyl(meth)acrylate compound, the alpha methylene lactone, and/or a mixture thereof to the weight percentage of the monomer is from about 1:12 to about 12:1, alternatively from about 1:10 to about 10:1, alternatively from about 1:5 to about 5:1, alternatively from about 12:1 to about 1:1, alternatively from about 10:1 to about 1:1 and alternatively from about 5:1 to about 1:1.

The first composition further may comprise at least one crosslinker having a molecular weight of a size suitable to penetrate the hair shaft and a molecular weight of 500 g/mole or less, alternatively from about 100 g/mole to about 500 g/mole, alternatively from about 100 g/mole to about 400 g/mole, and alternatively from about 200 g/mol to about 400 g/mole. Examples of crosslinkers suitable for use in the first composition of the present invention include, but are not limited to, 1,4-bisacryloylpiperazine, methylenebisacrylamide, ethylenebisacrylamide, divinylbenzene, poly-ethyleneglycol di(meth)acrylate, ethylene glycol di(meth)acrylate, 1,3-butanediol di(meth)acrylate, 1,4-butanediol di(meth)acrylate, neopentyl glycol di(meth)acrylate, and mixtures thereof.

In one embodiment, the ratio of the weight percentage of the sulfopropyl(meth)acrylate compound, the alpha methylene lactone, and/or the mixture thereof to the weight percentage of the crosslinker is from about 50:1 to about 10:1, alternatively from about 40:1 to about 10:1, and alternatively from about 20:1 to about 10:1.

The first composition further may comprise from about 0.01% to about 1% of at least one organic or inorganic catalyst. Non-limiting examples of suitable organic catalysts include 2-pyrrolidinoethanol, 1-piperidine-ethanol, 4-methylmorpholine, 2-morpholinoethanol, tetramethylethylenediamine, and mixtures thereof. Non-limiting examples of suitable inorganic catalysts include salts and/or hydrates of cerium(IV), cobalt(III), manganese(III), iron(III), nickel(II), copper(II), and mixtures thereof.

The present invention further may comprise, in some embodiments, a second composition comprising an initiator, useful for promoting binding of an ethylenic monomer and/or crosslinker to the keratin and/or to another monomer. The second composition may comprise from about 0.001% to about 5%, alternatively from about 0.01% to about 1%, and alternatively from about 0.1% to about 1%, of an initiator. Examples of suitable classes of initiators include, but are not limited to, peroxidisulfates, peroxides, peracids, phosphates, manganates, borates, bis-alkyamidines, sulfites, peroxyesters, bis-cyanocarboxylic acids, alpha-amino acetic acids, and mixtures thereof. Non-limiting examples of suitable initiators include sodium peroxydisulfate, 2,2′-azobis(2-methylpropionamidine)dihydrochloride, 2,2′-azobisisobutyronitrile, benzoyl peroxide, peracetic acid, ammonium cerium(IV) nitrate, and mixtures thereof. The second composition further may have a pH of above 7.0, alternatively of from about 7.0 to about 12.0, alternatively of from about 8.0 to about 12.0, and alternatively of from about 9.0 to about 11.0. In an alternative embodiment, the second composition has a pH of about 7.0 and below.

The present invention may comprise a third composition comprising a base, which is packaged separately from the first and the second composition, and which is useful for adjusting the pH of one, or a combination of any, of the compositions disclosed herein. The third, or pH adjusting, composition may have a pH of above 7.0, alternatively of from about 7.0 to about 12.0, alternatively of from about 8.0 to about 12.0, and alternatively of from about 9.0 to about 11.0. Additionally or alternatively, the third composition has a reserve alkalinity from about 1 to about 40, and alternatively from about 10 to about 30. Non-limiting examples of classes of suitable bases include, but are not limited to ammonium salts, amines, hydroxides, metasilicates, and mixtures thereof. Non-limiting examples of suitable bases include sodium hydroxide, potassium hydroxide, sodium metasilicate, ammonium hydroxide, ethanolamine, aminomethylpropanol, ammonium carbonate, and mixtures thereof.

The third composition further may comprise from about 0.1% to about 10%, and alternatively from about 0.5% to about 5%, of at least one organic or inorganic salt. Examples of organic salts include, but are not limited to, salts formed by reacting at least one anion chosen from phosphates, borates, silicates, bicarbonates, carbonates, chlorates, nitrates, and/or sulfonates, with at least one cation chosen from potassium, sodium, strontium, cadmium, calcium, ammonium (such as tetraalkylammonium and arylammonium), phosphonium, barium, lithium, and/or magnesium. Non-limiting examples of suitable organic salts include sodium monobutyl and dibutyl phosphates and sodium monoethyl and diethyl phosphates. In one embodiment, the salt comprises an inorganic cation. In one embodiment, the inorganic cation is a multivalent cation selected from the group consisting of magnesium, calcium, strontium, barium, copper, zinc, iron, nickel, cobalt, manganese, aluminum, silver, lanthanum, and complexes and mixtures thereof.

The present invention may comprise a fourth composition, useful for penetrating the hair shaft and reducing disulfide bonds. The fourth, or reducing, composition, comprises from about 1% to about 12%, alternatively from about 4% to about 10%, and alternatively from about 8% to about 10%, of a reducing agent. Examples of suitable reducing agents include, but are not limited to, sodium thioglycolate, anhydrous sodium thiosulfate, powdered sodium metabisulfite, thiourea, ammonium sulfite, thioglycolic acid, thiolacetic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulfoxylate, isooctyl thioglycolate, dl-cysteine, monoethanolamine thioglycolate, phosphines, and mixtures thereof.

### Dermatologically Acceptable Carrier

The compositions of the present invention may comprise from about 60% to about 99.9%, alternatively from about 70% to about 95%, and alternatively from about 80% to about 90%, of a dermatologically acceptable carrier. Carriers suitable for use with the composition(s) of the present invention include, for example, those used in the formulation of hair sprays, mousses, tonics, gels, and leave-on conditioners. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one embodiment, the carrier comprises water, fatty alcohols, volatile organic alcohols, and mixtures thereof.

### Optional Components

The composition(s) of the present invention may further comprise one or more optional components known or otherwise effective for use in hair care or personal care products, provided that the optional components are physically and chemically compatible with the essential components described herein, or do not otherwise unduly impair product stability, aesthetics, or performance. Non-limiting examples of such optional components are disclosed in International Cosmetic Ingredient Dictionary, Ninth Edition, 2002, and CTFA Cosmetic Ingredient Handbook, Tenth Edition, 2004, both of which are incorporated by reference herein in their entirety. Some non-limiting examples of such optional components are disclosed below, and include plasticizers, surfactants (which may be anionic, cationic, amphoteric or nonionic), neutralizing agents, propellants, hair conditioning agents (e.g., silicone fluids, fatty esters, fatty alcohols, long chain hydrocarbons, cationic surfactants, etc.), emollients, lubricants and penetrants such as various lanolin compounds, vitamins, proteins, preservatives, dyes, tints, bleaches, reducing agents and other colorants, sunscreens, thickening agents (e.g., polymeric thickeners, such as xanthan gum), physiologically active compounds for treating the hair or skin (e.g., anti-dandruff actives, hair growth actives), non-polymeric thickeners including clays, and perfume.

The composition(s) of the present invention further may comprise from about 0.1% to about 10%, and alternatively from about 0.2% to about 5.0%, of a gelling agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked allyl ether/acrylate copolymers (e.g., Salcare™ SC90 commercially available from Allied Colloids); unneutralized crosslinked copolymers of sodium polyacrylate, mineral oil, and PEG-1 trideceth-6 (e.g., Salcare™ SC91 commercially available from Allied Colloids); unneutralized crosslinked copolymers of methyl vinyl ether and maleic anhydride (e.g., Stabileze™ QM-PVM/MA copolymer commercially available from International Specialty Products); hydrophobically modified nonionic cellulose polymers; hydrophobically modified ethoxylate urethane polymers (e.g., Ucare™ Polyphobe Series of alkali swellable polymers commercially available from Union Carbide); and combinations thereof. In this context, the term “unneutralized” means that the optional polymer and copolymer gelling agent materials contain unneutralized acid monomers. Preferred gelling agents include water-soluble unneutralized crosslinked ethylene/maleic anhydride copolymers, water-soluble unneutralized crosslinked carboxylic acid polymers, water-soluble hydrophobically modified nonionic cellulose polymers and surfactant/fatty alcohol gel networks such as those suitable for use in hair conditioning products.

### II. Methods of Use

The present invention further describes methods of using the composition(s) of the present invention to chemically modify the internal portion of a hair shaft. The method comprises the step of applying to hair a first composition and an initiator as described herein. The initiator may form part of a second composition. The composition(s) may be applied by a variety of means, including with the fingers, hands, with an implement and/or with an energy delivery device. The composition(s) may be applied to wet hair or to dry hair. The amount applied will vary, and will depend upon the thickness and length of the hair, and the desired effect. In one embodiment, the composition(s) may be applied to substantially all of the hair, and alternatively to a portion of the hair. In one embodiment, the composition(s) may be applied to a portion of the hair that is proximal to the scalp, for example, from about 0 cm to about 10 cm, and alternatively from about 0 cm to about 5 cm proximal to the scalp. This may be desirable, for example, to “touch up” the roots of newly grown hair between treatments.

In one embodiment, the first composition comprising the monomer, and the second composition comprising the initiator are mixed prior to application to the hair, and applied to the hair as a single composition. For example, the first and second compositions may be mixed together one minute or less prior to application to the hair. Optionally, heat may be applied to the hair after applying the mixture. Alternatively, the first composition comprising the monomer is applied to the hair prior to application of the second composition comprising the initiator, without prior mixing. Alternatively, the second composition comprising the initiator is applied to the hair prior to application of the first composition comprising the monomer. The time interval between application of the first and of the second composition may vary, and may comprise from about 5 minutes to about 60 minutes, alternatively from about 15 minutes to about 45 minutes, and alternatively may be about 30 minutes. Optionally, heat may be applied to the hair for all or for a portion of the time period between application of the compositions.

A suitable initiator also may include light energy. Therefore, in one embodiment, the first composition may be applied to the hair, and the hair exposed to light energy. One non-limiting example includes allowing the composition to remain on the hair under a light source for about 5 minutes to about 30 minutes.

The method further may comprise the step of applying a third, or pH adjusting composition to the hair. The third composition may be mixed with the first and/or second composition, such that the final pH of the mixture of compositions is greater than 7.0, alternatively from 7.0 to about 12.0, alternatively from about 8.0 to about 12.0, and alternatively is from about 9.0 to about 11.0.

The method further may comprise the step of applying a fourth, or reducing, composition to the hair. In one embodiment, the reducing composition is applied prior to one or more compositions described herein and is allowed to remain on the hair from about 5 minutes to about 60 minutes, and preferably for about 15 minutes, after which time the composition is substantially removed from the hair, for example by rinsing with water. Optionally, heat may be applied to the hair for all or for a portion of the time during which the composition remains on the hair.

After treatment with one or more compositions of the present invention, the hair may exhibit one or more benefits, including but not limited to increased shape retention and/or durability, increased appearance of volume, increased resistance to the effects of humidity, for example, upon the style of the hair and/or upon the condition of the hair. Examples include all day hold of style, excellent curl definition, increased body and/or fullness, the ability to curl straight hair, and/or the ability to straighten curly hair.

### III. Kit

The present invention further describes a kit comprising a first composition and a second composition as described herein. The first and the second composition may be packaged in separate containers within the kit, and alternatively may be packaged in a single container which is capable of preventing admixing of the two compositions. The packaging may be of a size suitable for a single application, or unit dose, of the first and/or second composition. The kit may comprise a number of unit doses suitable for an indicated treatment regimen.

The kit further may comprise at least one additional composition selected from the group consisting of a pH adjustor, a reducing composition, and combinations thereof. In one embodiment, the pH adjustor has a reserve alkalinity as described herein and packaged in a container having a volume, such that when the contents of the container are mixed with the first and/or the second composition, the pH of the resulting mixture of compositions is greater than 7.0.

The kit further may comprise at least one additional component selected from the group consisting of a shampoo, a conditioner, a neutralizer, a colorant, a styling aid such as a gel, a mousse, a pomade, etc., an implement, an energy delivery device, instructions for complying with a treatment regimen, and combinations thereof. Examples of energy delivery devices include, but are not limited to light sources, including UV, visible light and infrared light, temperature change elements, hair dryers, heaters such as irons and heated curlers, ultrasonic devices, etc.

The kit further may comprise instructions for complying with a hair treatment regimen. The treatment regimen may comprise one or methods of use described herein, and may be directed toward treatment by a professional stylist or toward treatment by a consumer who is not a professionally-trained stylist.

### IV. Article of Commerce

The present invention further describes an article of commerce comprising a first composition and/or a second composition, as described herein, and a communication pertaining to the compositions. The communication may be printed material attached directly or indirectly to packaging, for example to a kit, that contains the compositions. Alternatively, the communication may be placed directly or indirectly near at least one composition. Alternatively, the communication may be an electronic or a broadcast message that is associated with the applicator and/or the composition. The communication may comprise images comparing the appearance of a person prior to use of the compositions to the appearance of the same person after use of the composition. One example of a suitable communication would be one directing a consumer having thin and/or limp hair to apply the composition(s) to the hair to increase the volume of hair and/or to create fuller looking hair for an extended period of time. Another example of a suitable communication would be one directing a professional hair stylist to apply the composition to clients desiring a longer lasting treatment to increase the volume of hair, the ease of styling, the retention of a style, etc.

### V. Method of Marketing

The present invention further comprises a method of marketing a kit comprising a first composition and a second composition as described herein, wherein the method of marketing comprises the step of making available to a consumer the kit, and providing a communication to the consumer that the compositions may provide one or more benefits to the hair, including but not limited to increased appearance of hair volume, increased resistance of the hair to moisture, increased ease of styling, and/or increased retention of a hair style. Examples include all day hold of style, excellent curl definition, increased body and/or fullness, the ability to curl straight hair, and/or the ability to straighten curly hair.

### EXAMPLES

### 1. The Following Describes One Non-Limiting Example of a Method of Using the Compositions of the Present Invention to Increase the Shape Retention and the Durability of Mammalian Hair:

Wash the hair or portion of hair to be treated with a cleansing shampoo. Thoroughly rinse and towel dry the hair, and comb the hair sufficiently to detangle. Apply a sufficient amount (estimated based upon the amount of hair to be treated), of a fourth composition, as described herein, to penetrate the hair shaft and to cleave disulfide bonds. Work the composition into hair with gentle messaging, avoiding pulling hair and avoiding contact of the composition with the skin and/or scalp and eyes. Optionally, the lotion may be evenly distributed by using a comb or other suitable implement. Allow the composition to remain on the hair, uncovered, for a period of time suitable to substantially cleave the disulfide bonds, as would be known by one of skill in the art such as a professional stylist. One example of a suitable time is 10 minutes at a temperature of 35° C.±5° C. Rinse the composition from the hair with warm water. Pat hair lightly with a towel until hair is damp and not dripping wet. Do not blow dry or comb hair.

Mix together by stirring or vigorously shaking, for about 30 seconds, about 50 ml of a first composition (Composition I—described below), about 1.0 ml of a second composition (Composition II—described below), and about 49 ml of a pH adjusting composition (Composition III—described below). Apply a suitable amount, as described above in this example, of the mixture to the damp hair. Gently massage the mixture into the hair, avoiding contact of the composition with the skin and/or scalp and eyes. Allow the composition to remain on the hair, uncovered, for about 30 minutes at 35° C.±5° C. Gently wash the hair (preferably with clarifying shampoo) and rinse thoroughly with water. Repeat the washing and rinsing once. Optionally, a conditioner may be applied to the hair. The hair may further be dried and styled as desired.

Composition I

| | | Example 1 | Example 2 | Example 3 | Example 4 | Example 5 | Example 6 |
|---|---|---|---|---|---|---|---|
| | Ingredients: | wt. % | wt. % | wt. % | wt. % | wt. % | wt. % |
| 1 | Purified Water | qs | qs | qs | qs | qs | qs |
| 2 | Cetyl Alcohol | 2.25 | — | 4.5 | 4.5 | — | 2.25 |
| 3 | Stearyl Alcohol | 2.25 | — | 4.5 | 4.5 | — | 2.25 |
| 4 | Hydroxypropylmethyl | — | 0.8 | — | 0.8 | 0.4 | — |
| | cellulose | | | | | | |
| 5 | Guar | — | 0.8 | — | — | 0.8 | — |
| | hydroxypropyltrimonium | | | | | | |
| | chloride | | | | | | |
| 6 | Polyquaterium-10 | — | — | — | — | 2.5 | 1 |
| 7 | Ceteareth 15 | 1.5 | — | 1.5 | 1.5 | — | 1.5 |
| 8 | Tetrasodium EDTA | 0.1 | 0.1 | 0.1 | 0.1 | 0.1 | 0.1 |
| 9 | Phenoxyethanol | 0.3 | — | 0.3 | 0.3 | — | 0.3 |
| 10 | Sodium Benzoate | 0.25 | 0.25 | 0.25 | 0.25 | 0.25 | 0.25 |
| 11 | 4-(2-Hydroxyethyl)- | — | 0.08 | — | — | — | — |
| | morpholine | | | | | | |
| 12 | 1,2-Octanediol | — | — | 2 | — | — | — |
| 13 | Isopropyl alcohol | — | — | 10 | — | — | — |
| 14 | Eldew ™ SL-205¹ | — | — | 2 | — | — | 2 |
| 15 | Methyl Paraben | — | 0.4 | — | — | 0.4 | — |
| | Monomer Actives: | | | | | | |
| 16 | Sulfopropylacrylate | 9.6 | 15 | 15 | 16 | 16 | 0.1 |
| | potassium salt | | | | | | |
| 17 | alpha-methylene-gamma- | 0.8 | — | 4 | — | 0.8 | — |
| | buyrolactone | | | | | | |
| 18 | Bisacryoylpiperizine | — | 1.5 | 1 | — | — | — |
| 19 | Methyl 2-acetamidoacrylate | — | 3.5 | — | — | — | — |
| | Fragrance: | | | | | | |
| 20 | Nourrissant 124 | — | 0.25 | 0.25 | 0.25 | 0.25 | 0.25 |

¹Isopropyl lauryl sarcosinate (Ajinomoto)

Mix the ingredients as follows, with continuous stirring. Add water to a suitable mixing vessel. Heat to about 80° C. Add ingredients 4, 5, 6, 8, and 10 and stir until dissolved. Add ingredients 2, 3, and 7, and allow temperature to again reach about 80° C. Add ingredients 9 and 11-15. Maintain temperature at about 80° C. for about 30 minutes. Cool to about 30° C. Add ingredients 16-20 and continue to stir until dissolved.

Composition II

| | | Example 1 | Example 2 | Example 3 | Example 4 | Example 5 | Example 6 |
|---|---|---|---|---|---|---|---|
| | Ingredients: | wt. % | wt. % | wt. % | wt. % | wt. % | wt. % |
| 21 | Sodium Persulfate | 100 | — | 90 | 10 | 50 | 1 |
| 22 | 2,2-Azobis(2- | — | 100 | — | — | — | — |
| | methylpropionamidine) | | | | | | |
| | dihydrochloride | | | | | | |
| 23 | Copper (III) Nitrate | — | — | 10 | — | — | — |

Combine all ingredients and mix well.

Composition III

| | | Example 1 | Example 2 | Example 3 | Example 4 | Example 5 | Example 6 |
|---|---|---|---|---|---|---|---|
| | Ingredients: | wt. % | wt. % | wt. % | wt. % | wt. % | wt. % |
| 24 | Purified Water | qs | qs | qs | qs | qs | qs |
| 25 | Ammonium Hydroxide (5N | 8 | — | — | 10 | 10 | — |
| | standard) | | | | | | |
| 26 | Sodium Hydroxide (1N | — | 10 | — | — | — | 1 |
| | standard) | | | | | | |
| 27 | Sodium Metasilicate | — | — | 10 | — | — | — |
| 28 | Barium Chloride | — | — | — | — | 8 | — |
| 29 | Strontium Nitrate | — | 8 | — | — | — | — |
| 30 | Lanthanum Nitrate | — | — | 2 | — | — | — |

Add water to a suitable mixing vessel. Add ingredients 25-30 and continue to stir until dissolved.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising from about 0.1% to about 20% of a sulfopropyl(meth)acrylate compound, an alpha methylene lactone, or a mixture thereof, wherein the sulfopropyl(meth)acrylate compound and the alpha methylene lactone each have a molecular weight of less than 500 grams/mole; and a dermatologically acceptable carrier.

2. The composition of claim 1, further comprising from about 0.1% to about 20% of at least one additional compound selected from the group consisting of an ethylenic monomer, a crosslinker, and mixtures thereof, wherein the additional compound has a molecular weight of less than 500 g/mole.

3. The composition of claim 1, further comprising from about 0.1% to about 20% each of an ethylenic monomer and a crosslinker, wherein the ethylenic monomer and the crosslinker each have a molecular weight of less than 500 g/mole.

4. The composition of claim 1, wherein the alpha methylene lactone is selected from the group consisting of alpha-methylene-gamma-butyrolactone, alpha-methylene-gamma-valerolactone, alpha-methylene-epsilon-caprolactone, 3-methylideneoxam-2-one, (5S)-S-(hydroxymethyl)-3-methylidene-oxolam-2-one, 3-methylidene-1-oxaspiro-[4,5]-decan-2-one and mixtures thereof.

5. The composition of claim 2, wherein the ethylenic monomer is selected from the group consisting of mesaconic acid, tert-2-pentenoic acid, tiglic acid, tiglic acid esters, furan-3-acrylic acid, 2-acrylamido-2-methyl-1-propanesulfonic acid, maleamic acid, 3-aminocrotonic acid, crotonic acid esters, itaconic anhydride, trimethylsilylacrylate, poly(ethyleneglycol)acrylates, N-vinylacetamide, 2-acetamidoacrylic acid, vinylsulfonic acid, tetrahydrofurfurylacrylate, N-methyl-N-vinylacetamide, vinylpropionate, vinylanisole, vinylcrotonate, methyl 3-hydroxy-2-methylenebutyrate, and mixtures thereof.

6. The composition of claim 2, wherein the crosslinker is selected from the group consisting of 1,4-bisacryloylpiperazine, methylenebisacrylamide, ethylenebisacrylamide, divinylbenzene, poly-ethyleneglycol di(meth)acrylate, ethylene glycol di(meth)acrylate, 1,3-butanediol di(meth)acrylate, 1,4-butanediol di(meth)acrylate, neopentyl glycol di(meth)acrylate, and mixtures thereof.

7. The composition of claim 2, wherein the second compound is an ethylenic monomer, and wherein the ratio of the weight percentage of the sulfopropyl(meth)acrylate compound, the alpha methylene lactone, or the combination thereof, to the weight percentage of ethylenic monomer is from about 1:12 to about 12:1.

8. The composition of claim 2, wherein the second compound is a crosslinker, and wherein the ratio of the weight percentage of the sulfopropyl(meth)acrylate compound, the alpha methylene lactone, or the combination thereof, to the weight percentage of crosslinker is from about 50:1 to about 10:1.

9. The composition of claim 1, wherein the pH of the composition is about 7.0 and below.

10. The composition of claim 1, further comprising from about 0.01% to about 0.1% of a catalyst.

11. The composition of claim 10, wherein the catalyst is selected from the group consisting of 2-pyrrolidinoethanol, 1-piperidine-ethanol, 4-methylmorpholine, 2-morpholinoethanol, tetramethylethylenediamine, salts and/or hydrates of cerium(IV), cobalt(III), manganese(III), iron(III), nickel(II), copper(II), and mixtures thereof.

12. A method of chemically modifying the internal region of a hair shaft, comprising the step of applying to hair a first composition comprising:
a) from about 0.1% to about 20% of a sulfopropyl(meth)acrylate compound, an alpha methylene lactone, or a mixture thereof, wherein the sulfopropyl(meth)acrylate compound and the alpha methylene lactone each have a molecular weight of less than 500 grams/mole; and a dermatologically acceptable carrier; and
b) applying to the hair a second composition comprising an initiator.

13. The method of claim 12, wherein a second composition comprises the initiator.

14. The method of claim 12, wherein the initiator is light energy.

15. The method of claim 14, wherein the hair to which the first composition has been applied is exposed to the light energy for a period of from about 5 minutes to about 30 minutes.

16. The method of claim 12, further comprising the step of applying to the hair a composition comprising a reducing agent.

17. The method of claim 16, wherein the reducing agent is selected from the group consisting of sodium thioglycolate, anhydrous sodium thiosulfate, powdered sodium metabisulfite, thiourea, ammonium sulfite, thioglycolic acid, thiolacetic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulfoxylate, isooctyl thioglycolate, dl-cysteine, monoethanolamine thioglycolate, phosphines, and mixtures thereof.

18. The method of claim 13, wherein the initiator is selected from the group consisting of peroxidisulfates, peroxides, peracids, phosphates, manganates, borates, bis-alkyamidines, sulfites, peroxyesters, bis-cyanocarboxylic acids, alpha-amino acetic acids, sodium peroxydisulfate, 2,2′-azobis(2-methylpropionamidine)dihydrochloride, 2,2′-azobisisobutyronitrile, benzoyl peroxide, peracetic acid, ammonium cerium(IV) nitrate, and mixtures thereof.

19. The method of claim 12, wherein the first composition has a pH of about 7.0 and below.

20. The method of claim 13, wherein the first and the second compositions are mixed prior to application to the hair.

21. The method of claim 12, further comprising the step of applying heat to the hair.

22. The method of claim 13, wherein the second composition is applied to the hair after the first composition.

23. The method of claim 22, wherein the second composition is applied from about 5 minutes to about 60 minutes after the first composition.

24. The method of claim 13, wherein the first composition is applied to the hair after the second composition.

25. The method of claim 24, wherein the first composition is applied from about 5 minutes to about 60 minutes after the second composition.

26. The method of claim 13, wherein the first composition and the second composition are applied to a portion of the hair.

27. The method of claim 26, wherein the portion of the hair is proximal to the scalp.

28. The method of claim 12, wherein after chemical modification, the hair exhibits at least one benefit selected from the group consisting of increased style retention, increased style durability, increased appearance of volume, increased resistance to moisture, and combinations thereof.

29. The method of claim 12, wherein after chemical modification, the bending rigidity of the hair is at least 20% greater than the bending rigidity of the same hair prior to chemical modification.

30. The method of claim 12, wherein after chemical modification, the average curl retention number of the hair is at least 20% less than the average curl retention number of the same hair prior to chemical modification.

31. The method of claim 12, wherein the alpha methylene lactone is selected from the group consisting of alpha-methylene-gamma-butyrolactone, alpha-methylene-gamma-valerolactone, alpha-methylene-epsilon-caprolactone, 3-methylideneoxan-2-one, (5S)-S-(hydroxymethyl)-3-methylidene-oxolan-2-one, 3-methylidene-1-oxaspiro-[4,5]-decan-2-one, and mixtures thereof.

32. The method of claim 12, wherein the composition further comprises from about 0.1% to about 20% of at least one additional compound selected from the group consisting of an ethylenic monomer, a crosslinker, and mixtures thereof, wherein the additional compound has a molecular weight of less than 500 g/mole.

33. The method of claim 32, wherein the ethylenic monomer is selected from the group consisting of mesaconic acid, tert-2-pentenoic acid, tiglic acid, tiglic acid esters, furan-3-acrylic acid, 2-acrylamido-2-methyl-1-propanesulfonic acid, maleamic acid, 3-aminocrotonic acid, crotonic acid esters, itaconic anhydride, trimethylsilylacrylate, poly(ethyleneglycol)acrylates, N-vinylacetamide, 2-acetamidoacrylic acid, vinylsulfonic acid, tetrahydrofurfurylacrylate, N-methyl-N-vinylacetamide, vinylpropionate, vinylanisole, vinylcrotonate, methyl 3-hydroxy-2-methylenebutyrate, and mixtures thereof.

34. The method of claim 32, wherein the crosslinker is selected from the group consisting of 1,4-bisacryloylpiperazine, methylenebisacrylamide, ethylenebisacrylamide, divinylbenzene, poly-ethyleneglycol di(meth)acrylate, ethylene glycol di(meth)acrylate, 1,3-butanediol di(meth)acrylate, 1,4-butanediol di(meth)acrylate, neopentyl glycol di(meth)acrylate, and mixtures thereof.

35. The method of claim 32, wherein the additional component is an ethylenic monomer, and wherein the ratio of the weight percentage of the sulfopropyl(meth)acrylate compound, the alpha methylene lactone, or the combination thereof to the weight percentage of ethylenic monomer is from about 1:12 to about 12:1.

36. The method of claim 32, wherein the additional component is a crosslinker and wherein the ratio of the weight percentage of the sulfopropyl(meth)acrylate compound, the alpha methylene lactone, or the combination thereof to the weight percentage of crosslinker is from about 50:1 to about 10:1.

37. The method of claim 12, wherein the first composition further comprises from about 0.1% to about 1% of a catalyst.

38. The method of claim 37, wherein the catalyst is selected from the group consisting of 2-pyrrolidinoethanol, 1-piperidine-ethanol, 4-methylmorpholine, 2-morpholinoethanol, tetramethylethylenediamine, salts and/or hydrates of cerium(IV), cobalt(III), manganese(III), iron(III), nickel(II), copper(II), and mixtures thereof.

39. A kit comprising:
a) a first composition comprising from about 0.1% to about 20% of a sulfopropyl(meth)acrylate compound, an alpha methylene lactone, or a mixture thereof, wherein the sulfopropyl(meth)acrylate compound and the alpha methylene lactone each have a molecular weight of less than 500 grams/mole; and a dermatologically acceptable carrier; and
b) a second composition comprising an initiator;
wherein the first composition and the second composition are separately packaged.

40. The kit of claim 39, further comprising a pH adjusting composition.

41. The kit of claim 40, wherein the pH adjusting composition comprises from about 0.1% to about 10% of at least one multivalent cation.

42. The kit of claim 41, wherein the multivalent cation is selected from the group consisting of magnesium, calcium, strontium, barium, copper, zinc, iron, nickel, cobalt, manganese, aluminum, lanthanum, silver, and complexes and mixtures thereof.

43. The kit of claim 40, wherein the pH adjusting composition has a reserve alkalinity of from about 1 to about 40.

44. The kit of claim 39, further comprising a composition comprising a reducing agent.

45. The kit of claim 44, wherein the reducing agent is selected from the group consisting of sodium thioglycolate, anhydrous sodium thiosulfate, powdered sodium metabisulfite, thiourea, ammonium sulfite, thioglycolic acid, thiolacetic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulfoxylate, isooctyl thioglycolate, dl-cysteine, monoethanolamine thioglycolate, phosphines, and mixtures thereof.

46. The kit of claim 39, further comprising at least one additional component selected from the group consisting of a shampoo, a conditioner, a colorant, a styling aid such as a gel, a mousse, a pomade, an implement, an energy delivery device, instructions for complying with a treatment regimen, and combinations thereof.

47. The kit of claim 39, wherein the first composition has a pH of about 7.0 and below.

48. The kit of claim 46, wherein the treatment regimen comprises the step of mixing together the first composition and the second composition prior to application to the hair.

49. The kit of claim 46, wherein the treatment regimen comprises the step of mixing together the first composition, the second composition and the pH adjusting composition prior to application to hair.

50. The kit of claim 46, wherein the treatment regimen comprises the step of mixing together the first composition, the second composition, the pH adjusting composition and the reducing composition prior to application to hair.

51. The kit of claim 46, wherein the treatment regimen comprises the step of applying the first composition to the hair prior to applying the second composition to the hair.

52. The kit of claim 51, wherein the treatment regimen comprises the step of waiting from about 5 minutes to about 60 minutes after application of the first composition before applying the second composition.

53. The kit of claim 52, wherein the treatment regimen comprises the step of waiting about 30 minutes after application of the first composition before applying the second composition.

54. The kit of claim 46, wherein the treatment regimen comprises the step of applying the second composition to the hair prior to applying the first composition to the hair.

55. The kit of claim 54, wherein the treatment regimen comprises the step of waiting from about 5 minutes to about 60 minutes after application of the second composition before applying the first composition.

56. The kit of claim 55, wherein the treatment regimen comprises the step of waiting about 30 minutes after application of the second composition before applying the first composition.

57. The kit of claim 39, wherein the first composition further comprises from about 0.1% to about 20% of at least one additional compound selected from the group consisting of an ethylenic monomer, a crosslinker, and mixtures thereof, wherein the additional compound has a molecular weight of less than 500 g/mole.

58. The kit of claim 57, wherein the ethylenic monomer is selected from the group consisting of mesaconic acid, tert-2-pentenoic acid, tiglic acid, tiglic acid esters, furan-3-acrylic acid, 2-acrylamido-2-methyl-1-propanesulfonic acid, maleamic acid, 3-aminocrotonic acid, crotonic acid esters, itaconic anhydride, trimethylsilylacrylate, poly(ethyleneglycol)acrylates, N-vinylacetamide, 2-acetamidoacrylic acid, vinylsulfonic acid, tetrahydrofurfurylacrylate, N-methyl-N-vinylacetamide, vinylpropionate, vinylanisole, vinylcrotonate, methyl 3-hydroxy-2-methylenebutyrate, and mixtures thereof.

59. The kit of claim 57, wherein the crosslinker is selected from the group consisting of 1,4-bisacryloylpiperazine, methylenebisacrylamide, ethylenebisacrylamide, divinylbenzene, poly-ethyleneglycol di(meth)acrylate, ethylene glycol di(meth)acrylate, 1,3-butanediol di(meth)acrylate, 1,4-butanediol di(meth)acrylate, neopentyl glycol di(meth)acrylate, and mixtures thereof.

60. The kit of claim 57, wherein the additional component is an ethylenic monomer, and wherein the ratio of the weight percentage of the sulfopropyl(meth)acrylate compound, the alpha methylene lactone, or the combination thereof to the weight percentage of ethylenic monomer is from about 1:12 to about 12:1.

61. The kit of claim 57, wherein the additional component is a crosslinker and wherein the ratio of the weight percentage of the sulfopropyl(meth)acrylate compound, the alpha methylene lactone, or the combination thereof to the weight percentage of crosslinker is from about 50:1 to about 10:1.

62. The kit of claim 39, wherein the first composition further comprises from about 0.1% to about 1% of a catalyst.

63. The kit of claim 62, wherein the catalyst is selected from the group consisting of 2-pyrrolidinoethanol, 1-piperidine-ethanol, 4-methylmorpholine, 2-morpholinoethanol, tetramethylethylenediamine, salts and/or hydrates of cerium(IV), cobalt(III), manganese(III), iron(III), nickel(II), copper(II), and mixtures thereof.

64. The kit of claim 39, wherein the alpha methylene lactone is selected from the group consisting of alpha-methylene-gamma-butyrolactone, alpha-methylene-gamma-valerolactone, alpha-methylene-epsilon-caprolactone, 3-methylideneoxan-2-one, (5S)-S-(hydroxymethyl)-3-methylidene-oxolan-2-one, 3-methylidene-1-oxaspiro-[4,5]-decan-2-one, and mixtures thereof.

65. The kit of claim 39, wherein the initiator is selected from the group consisting of peroxidisulfates, peroxides, peracids, phosphates, manganates, borates, bis-alkyamidines, sulfites, peroxyesters, bis-cyanocarboxylic acids, alpha-amino acetic acids, sodium peroxydisulfate, 2,2′-azobis(2-methylpropionamidine)dihydrochloride, 2,2′-azobisisobutyronitrile, benzoyl peroxide, peracetic acid, ammonium cerium(IV) nitrate, and mixtures thereof.

66. The kit of claim 46, wherein the instructions indicate that the treatment regimen results in at least one benefit to the hair to which the compositions are applied, selected from the group consisting of increased style retention, increased style durability, increased appearance of volume, increased resistance to moisture, and combinations thereof.
